(19) United States
(12) Patent Application Publication
(10) Pub. No.: US 2025/0143642 A1
(43) Pub. Date: May 8, 2025

(54) AN ADHESIVE SKIN PATCH AND A METHOD FOR MANUFACTURING IT

(71) Applicant: TEKNOLOGIAN TUTKIMUSKESKUS VTT OY, Espoo (FI)

(72) Inventors: Mohammadhossein BEHFAR, Espoo (FI); Olli-Heikki HUTTUNEN, Espoo (FI); Johanna HIITOLA-KEINÄNEN, Espoo (FI)

(21) Appl. No.: 18/570,002

(22) PCT Filed: Jun. 15, 2022
(86) PCT No.: PCT/FI2022/050417

§ 371 Date: Dec. 13, 2023

(30) Foreign Application Priority Data
Jun. 16, 2021 (FI) 20215709

(51) Int. Cl. A61B 5/00; A61B 5/263; A61B 5/28

(52) U.S. Cl. CPC A61B 5/6833; A61B 5/263; A61B 5/28

(57) ABSTRACT

An adhesive skin patch and a method that enables provision of an adhesive skin patch which is very thin, conforms easily to deformations of the skin and can still be manufactured in a sequentially progressing industrial process that can be automated for commercial production. The adhesive skin patch includes conductive parts that provide contact to the skin and access for external electrical contacts.

### FIELD OF THE DISCLOSURE

This text relates to printed electronics, and particularly to an improved adhesive skin patch and an improved method for manufacturing it.

### BACKGROUND

Use of monitoring applications that process information from direct skin-sensor interactions is continuously increasing. It is already quite common to continuously monitor the quality of one's sleep, physical condition, or effectiveness of exercise, for example. Diagnostic applications attempt to observe selected vital signals of monitored subjects for longer periods outside laboratory settings. For these purposes, different kinds of sensors and electrodes have been planted into watches, rings, straps and belts, for example.

One important type of on-skin devices is adhesively attached and removable skin patches, which have been used already for some time in medical applications. However, reasonably priced and comfortably worn skin patches for frequent and/or long-term use have not yet been available. Many commercially available skin patches still include a stiff substrate sheet that feels uncomfortable when glued on the skin, specifically for a longer time. They also tend to include components that protrude outwards and cannot be discreetly hidden under one's clothing. At least some of the components or connections are even exposed so that the patch cannot be safely used in humid or wet conditions.

Some more recent solutions apply skin patches that do not include the conventional reinforcing substrate but are formed of a hybrid combination of rigid components integrated with flexible ones. However, they also suffer from same lack of comfort in use and due to the level of complex integration, they tend to be relatively expensive to manufacture. Such hybrid solutions are not really suitable for use in applications where the skin patch needs to be comfortably used, easily changed and disposed after its use.

Epidermal electronics is a new class of wearable technology where ultrathin, light-weight and flexible, or even stretchable, devices are mounted on a skin with a conformal contact. However, despite considerable advances in research, several limiting factors have hindered the exploitation of epidermal electronics in practical applications. When in use, skin patches are exposed to mechanical deformations and humidity changes, and it is a complex technical challenge to provide a structure that is thin and flexible and at the same appropriately protects conductive parts from humidity. Additionally, so far only customized fabrication methods have been used to produce the demonstrated samples and connectivity issues have remained completely unsolved. Even if images of very thin patches with underlying skin contacts already appear in publications, the output connections shown rely only on additional, external wiring arrangements.

### BRIEF DESCRIPTION OF THE DISCLOSURE

An object of the present disclosure is to provide an adhesive skin patch and a method that enables provision of an adhesive skin patch which is very thin, conforms easily to deformations of the skin and can still be manufactured in a sequentially progressing industrial process that can be automated for commercial production.

The object of the invention is achieved by the adhesive skin patch and the method, which are characterized by what is stated in the independent claims. Exemplary embodiments of the disclosure are disclosed in the dependent claims.

### BRIEF DESCRIPTION OF THE DRAWINGS

In the following examples for implementing of the invention will be described in greater detail with reference to the accompanying drawings, in which

FIGS. 1A to 1C illustrate example layer structures for an exemplary adhesive skin patch;

FIG. 2 shows a top view of an on-skin part of the adhesive skin patch;

FIG. 3 illustrates stages of a method for creating the layered structure for the adhesive skin patch;

FIG. 4 includes three images that show an exemplary skin patch formed with the layered structure; and

FIG. 5 illustrates an advantageous use case for an adhesive skin patch;

FIGS. 6A to 6C illustrate layers of an on-skin part in exemplary multi-layer stacks for a skin patch; and

FIGS. 7A and 7B illustrate layers of an on-skin part in further exemplary multi-layer stacks for a skin patch.

### DETAILED DESCRIPTION

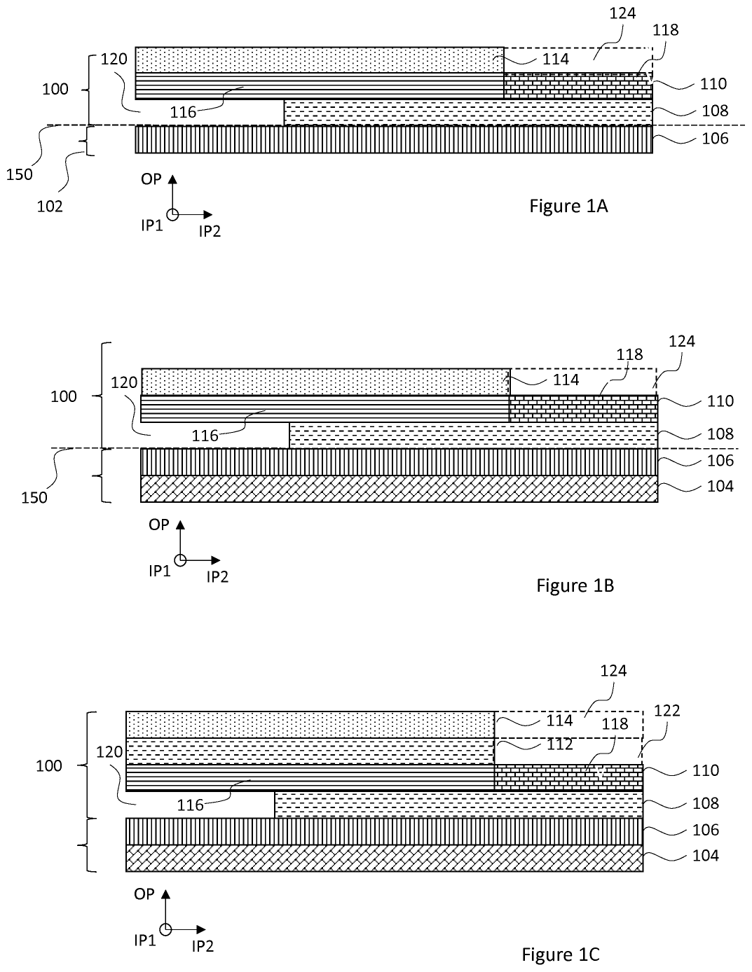

FIGS. 1A to 1C illustrate example layer structures for an adhesive skin patch that is applicable as a wearable electronic device or a part of a wearable electronic device that collects information on a subject through a contact placed on the skin of the subject.

An adhesive patch that settles conformably on the skin needs to be so thin that in practise it cannot be manufactured or transferred on the skin without some reinforcing support structure. The layer stack of FIGS. 1A to 1C thus shows an on-skin part 100 and a releasable part 102, wherein the on-skin part 100 forms the skin patch that remains attached to the skin of a subject during use, and the releasable part 102 can be removed from the layer stack after the on-skin part 100 has been safely and conformably attached on the skin. The releasable part provides the necessary reinforcement for the various manufacturing and transfer stages that precede the actual use of the adhesive patch.

The term layer in this text refers to a thickness of deposited material that fully or partially covers an underlying surface, which in turn is formed by one or more previously deposited layers. The underlying surface may be planar if, for example, it is provided by a planar substrate sheet, or it may be curved resulting from patterns or formations in previously deposited layers. In sequentially progressing deposition stages, a presently deposited layer tends to conform to the form of the underlying surface and, for example, fill underlying patterned recesses, if such exist in the underlying layer.

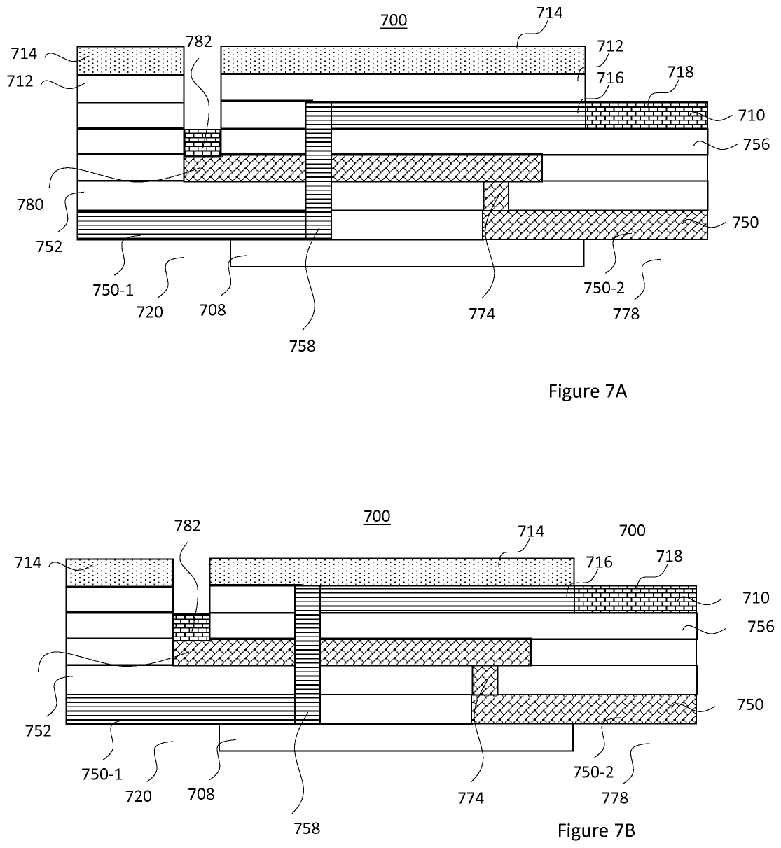

The term connector in this text refers to a conductive element that includes a connector region or a combination of a connector region and a sensing region. The connector may be implemented as one layer pattern or as a pattern that includes two layers, layer for a connector region and a layer for a sensing region. FIGS. 1A to 6B show examples wherein a connector is provided as a conductive layer pattern. FIG. 7 shows an example wherein regions of the connector are implemented with two layer patterns, one stacked on the other.

FIG. 1A shows schematically an example of a basic layer stack for a skin patch. In this example, the releasable part 102 includes a layer of liquid-soluble polymer 106. The liquid-soluble polymer 106 is a substance that functions in the layer stack as a carrier for the on-skin part 100. The liquid-soluble polymer 106 is a polymer material that is sturdy enough to act as a substrate for an accumulating web or sheet that is formed in layer deposition steps of an industrial manufacturing process. It remains attached to the on-skin part 100 during fabrication and transfer stages but can be easily dissolved when the on-skin part to the skin has been attached onto the skin. Advantageously, the liquid-soluble polymer is soluble with water so that the layer can be very easily washed off after the on-skin part has been placed on the skin. However, polymers soluble with other liquid solvents may be applied within the scope. The layer of liquid soluble polymer 106 is configured to be considerably less elastic than the on-skin part so that it does not essentially stretch but provides mechanical support to the on-skin patch and thus prevents wrinkling or curing of the ultra-thin part during the application stage. For example, the material composition of the liquid soluble polymer may be selected to provide the required stiffness and/or the layer of liquid-soluble polymer 106 may be considerably thicker than the on-skin part 100.

FIG. 1B shows schematically an alternative layer stack, in which the releasable part is a dual-layer combination that includes a further support layer 104. This dual-layer structure is advantageous for implementations where mechanical stress on the layer stack at some stage is high, for example when the layer stack is manufactured by roll-to-roll processing. The support layer 104 is thus advantageously made of a material that is flexible enough for roll-to-roll processing but sturdy enough to act as a substrate for an accumulating web or sheet that is formed in layer deposition steps of an industrial manufacturing process. The support layer 104 can also be used to protect the other parts of the skin patch on its way to the user. For example, the support layer 104 may be designed to be removed from the layer of liquid soluble polymer 106 before the skin patch is attached onto the skin, so that the at the time of application, the skin patch includes only the layer stack of FIG. 1A. Alternatively, the support layer 104 may be designed to be removed from the layer of liquid soluble polymer 106 after the skin patch is attached onto the skin so that both the layer of liquid soluble polymer 106 and the support layer 104 protect the thin and easily crumpling on-skin part 100 until it is safely attached onto the skin. A variety of materials can be used for the support layer, examples of such materials include polymers, paper, cardboard and the like. Advantageously, the support layer is made of polyethylene terephthalate (PET) that suits very well for this purpose because of its material characteristics. PET films adapt well to roll-to-roll processing, and a PET film that is elastic enough for roll-to-roll processing still supports the softer and more elastic parts of the layer stack very well. A PET film is also easy to use when the support layer needs to be detached from the layer of liquid soluble polymer 106.

The on-skin part 100 is the skin patch that in its basic form includes an adhesive layer, an elastic layer and a conductive layer enclosed between them. FIGS. 1A and 1B show an example of an on-skin part 100 that includes a first layer of polysiloxane 108; a conductor in form of a conductive layer 110, and an adhesive layer 114. The term polysiloxane refers here to polymerized siloxanes, also known as silicones. Silicones exhibit many properties that make them useful for use in skin patches, for example, low thermal conductivity, low chemical reactivity, low toxicity, thermal stability, electrical insulation, to mention some. In the context of very thin skin patches, it is especially advantageous that a selected polysiloxane can be deposited as a fluid onto an underlying layer stack to create a patterned form and then cured to function as an elastic solid. The term elastic solid refers here to polymers that have a solid form but provide rubber-like elasticity. An advantageous type of polysiloxanes for these example embodiments covers silicone compositions that include polydimethylsiloxane (PDMS), which is also known as dimethylpolysiloxane or dimethicone. The mechanical properties of PDMS are affected by a variety of factors, which can be decided before PDMS is cured so they are relatively easy to tune to function after curing as an elastic solid. Due to the viscoelastic properties of PDMS before curing, it also adapts well for deposition onto an accumulating web or sheet of a sequentially progressing industrial process.

The adhesive layer may be provided with any type of conformal skin-friendly adhesive selected, for example, from the commercially available acrylic based skin adhesives and silicone based skin adhesives. The essential requirement for the embodiments of FIGS. 1A and 1B is that the material of the adhesive layer can be formed into an elastic layer structure that attaches to the skin but maintains its patterned form during use.

FIG. 1C shows an example of an on-skin part 100 that includes a first layer of polysiloxane 108; a conductive layer 110, a second layer of polysiloxane 112, and an adhesive layer 114. The second layer of polysiloxane may be included in the layer stack to provide an additional protective and/or electrically insulating layer between the conductive layer and the adhesive layer.

In all examples of FIGS. 1A to 1C, the conductor is a conductive layer 110 formed into a pattern that comprises a conductor region 116, and a sensing region 118 that is in electrical connection with the conductor region 116. In some applications, the conductor region 116 and the sensing region 118 may at least partially overlap. As can be seen from all FIGS. 1A to 1C, the first layer of polysiloxane 108 includes an opening 120 to at least part of the conductor region 116. The expression “opening to” in this context can be interpreted by considering that the layer on which the on-skin part has been deposited forms a planar reference plane 150 that extends in two mutually orthogonal in-plane directions IP1 and IP2, and the layer stack is grown in an out-of-plane direction OP, which is orthogonal to the two in-plane directions, as shown in FIGS. 1A to 1C. An opening in one layer to a part in another layer means that the opening is a discontinuity in the layer thickness, and a projection of said opening and a projection of said part in the other layer in the reference plane at least partially coincide. In a sequentially progressing industrial layer deposition process, an opening in an underlying layer typically becomes at least partially filled with substance of the next deposited layer. This means that in practise the resulting structure does not substantially include void spaces and the term opening does not in this context refer to an empty recess.

Accordingly, the opening 120 in FIG. 1A is a discontinuity in the layer structure of the first layer of polysiloxane 108, and a projection of the opening 120 and a projection of the conductor region 116 in the reference plane at least partially coincide. When the layer of the conductor region is deposited, it at least partially fills the opening 120 and can bring the surface of the conductive part locally in level with the side of the first layer of polysiloxane 108. When the liquid-soluble polymer 106 of the releasable part is removed, the first layer of polysiloxane 108 and also this locally exposed part of the conductor region 116 is exposed. Accordingly, the aspect of layered structures is innovatively taken into use to provide a contact for an external electrical connection. During layer deposition, the fluidic material of the conductor region 116 at least partially fills the opening 120 and therefore provides an open interface for a conductive path to the sensing region 118. This interface for external electrical connection becomes available for use without further operations or additional wiring arrangements when the liquid-soluble polymer 106 is dispersed with solvent from the surface of the layer stack. As will be described in more detail later on, the opening 120 can be provided with conventional patterning means in a sequentially progressing industrial process, but due to the specific layered stack structure presented herein, it provides an output for electrical information available through the skin contact of the sensing region 118.

The sensing region 118 represents here part of a layer pattern that includes material or a structured combination of materials, wherein some electrical characteristic of that part changes as a function of a change detectable on the skin of the subject. Such change may be, for example, a change in surface potential, chemical change, temperature change, or mechanical change (e.g. strain). In the example of FIG. 1, the sensing region includes a combination of silver/silver chloride paste (Ag/AgCl) that is responsive to biopotentials generated by excitable cells. This response can be detected as a measurable signal with the external electrical connection. Examples of measurable biopotential signals include of electrocardiograms (ECG), electromyograms (EMG), electroencephalograms (EEG), electrooculograms (EOG), to name some.

As shown in in FIGS. 1A and 1B, the adhesive layer 114 includes an opening 124 to at least part of the sensing region 118. Again, this expression means that the opening 124 is a discontinuity in the layer thickness of the adhesive layer 114, and a projection of the opening 124 and a projection of the sensing region 118 in the reference plane 150 at least partially coincide. The role of the opening 124 is to leave the sensing region 118 exposed on the skin side. The adhesive layer 114 is very thin, the whole layer structure of the on-skin part 100 is very flexible and conformal, and the skin of a person is very flexible, so in practise this opening does not form a void space between the skin and the sensing region 118, but the sensing region presses in use against the skin so that a direct skin contact can be achieved for measurements with the sensing region 118.

In the configuration of 1C, the second layer of polysiloxane 112 is between the conductive layer and the adhesive layer 114 and includes also an opening 122 to at least part of the sensing region 118. The opening 122 is thus a discontinuity in the layer thickness of the second layer of polysiloxane 112, and a projection of the opening 122 and a projection of the sensing region 118 in the reference plane at least partially coincide. The role of the opening 122 is to combine with the opening 124 on the adhesive layer 114 to expose the sensing region 118 on the skin side. The second layer of polysiloxane 112 is very thin, the whole layer structure of the on-skin part 100 is very flexible and conformal, and the skin of a person is very flexible, so in practise the openings in the second layer of polysiloxane 112 and the adhesive layer do not form a void space between the skin and the sensing region 118, but the sensing region presses in use against the skin so that a direct skin contact can be achieved for measurements.

As can be seen from FIG. 1C, notwithstanding the parts of the conductive layer 110 that are deliberately left exposed through the openings 120, 122, the rest of the conductive layer 110 is tightly pressed between the first and second layers of polysiloxane 108, 112. This means that in regions beyond the openings 120, 122, the first layer of polysiloxane and the second layer of polysiloxane enclose the conductive layer in a water-tight manner. By reducing exposure to changes in humidity, this packaged layer structure further improves stability of operation of the measurements.

For the required conformity, thickness of the adhesive layer is preferably less than 100 μm, even more preferably between 5 to 15 μm. Thickness of the conductive layer is preferably less than 30 μm, even more preferably between 5 to 15 μm. Thickness of the layers of polysiloxane is preferably less than 100 μm, even more preferably between 5 to 15 μm. Thickness of the conductive layer is preferably less than 30 μm, even more preferably between 5 to 15 μm. Thickness of the layer of liquid soluble polymer is preferably less than 200 μm, even more preferably between 50 to 100 μm. The thickness of the support layer is preferably less than 200 μm, even more preferably between 20 to 50 μm.

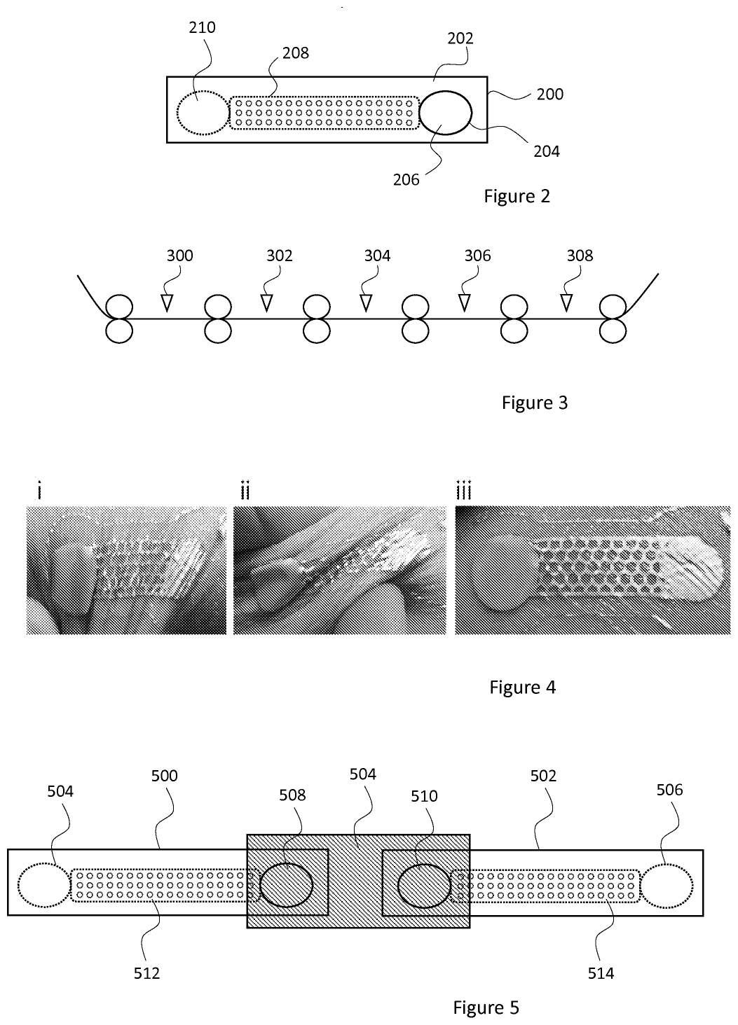

FIG. 2 shows a top view of the on-skin part 200 of an adhesive skin patch illustrated with the layer structure of FIG. 1A to 1C after removal of the releasable part. The structure is described with reference to FIG. 1C, but as already explained, the skin patch does not necessarily include the second layer of polysiloxane, The exterior of the on-skin part 200 is formed of a surface of the first layer of polysiloxane 202 that includes an opening 204. A first part 206 of a conductor region is accessible for external electrical connections through the opening 204 in the first layer of polysiloxane 202. The second part 208 of the conductor region is tightly enclosed under exterior of the on-skin part 200 between the first layer of polysiloxane 202 and a second layer of polysiloxane or an adhesive layer under it. The second part 208 of the conductor region ends up to the sensing region 210 that is also safely secured under the first layer of polysiloxane 202 but exposed to the underlying skin through the opening in the adhesive layer or through the common opening in the second layer of polysiloxane and the adhesive layer that binds the second layer of polysiloxane to the underlying skin. The layer structure described with FIGS. 1 and 2 shows how the conductive path between the first part 206 of the conductor region and the sensing region is provided in integrated form, without additional external wiring arrangements or other complex procedures during manufacture. It is noted that the forms and patterns shown in FIG. 2 are exemplary only. A broad variety of electrode and conductor forms may be applied in the pattern of the conductor.

FIG. 3 illustrates stages of a method for creating the layered structure for the adhesive skin patch described with FIGS. 1C and 2. Additional details for description of FIG. 3 may be referred from FIGS. 1 and 2, and vice versa. FIG. 3 is a schematic drawing that presents the stages and their order in a simplified form, the arrangement of reels, motors and mechanisms for rolling the reels as such is well known to a person skilled in the art printed electronics and will not be discussed in more detail herein.

In the method, the web to be worked on is based on a support layer carrier that is reeled to the process. Advantageously the carrier is made of polyethylene terephthalate (PET) that even in the sub-millimetre thicknesses of the order of 0.01-0.5 millimetre adapts well to roll-to-roll reeling and provides a robust and protective basis for the subsequent deposition stages. A layer of liquid-soluble polymer is then deposited (stage 300) on a surface of the carrier. The liquid-soluble polymer may be applied on the surface of the of the carrier in liquid form and may need to be cured before the subsequent deposition stage. Curing may include drying, heat treatment, chemical treatment, or the like.

In the next stage, a first layer of polysiloxane is deposited (stage 302) on the layer of liquid-soluble polymer. The polysiloxane may be, for example, polydimethylsiloxane based material. The first layer of polysiloxane includes at least one opening so it needs to be deposited in a patterned form. Advantageously, pattering is implemented by masking the part of the underlying surface of the liquid-soluble polymer that is not intended to be covered with polysiloxane with a protective mask film, like a PET. The protective mask film is typically removed before the polysiloxane is cured for the next deposition stage. Other methods for patterning, like slot die coating or screen printing may be applied without deviating from the scope of protection.

In the next stage, a conductive layer pattern that includes a conductor region and a sensing region is deposited (stage 304) on the first layer of polysiloxane. It should be noted that FIG. 3 is schematic. Depending on the selected patterning method and materials, the deposition step 304 may be implemented in one process stage or it may include more than one substages, for example, a sub-stage for depositing the conductor region with one material and another sub-stage for depositing the sensing region with another material. Advantageously, the conductor region is made of a highly conductive material, like copper or silver. The sensing region may include a volume of a material or a combination of materials, and electrical characteristics of this volume of material/materials changes as a function of a change detectable on the skin of the subject. The sensing region may alternatively include a structured combination of materials, electrical characteristics of which are responsive to a detectable change on the skin of the subject. There are many known methods to deposit a conductive pattern on a progressing sheet or web, for example, screen printing, stencil printing. In the deposition, the pattern of the conductive layer is positioned so that the conductor region of the pattern is aligned to at least partly coincide with the opening in the first layer of polysiloxane. This means that during deposition, the deposited material at least partially fills the opening in the underlying first layer of polysiloxane.

In the next stage, a second layer of polysiloxane is deposited (stage 306) on the conductive layer. Also the second layer of polysiloxane includes at least one opening so it needs to be deposited in a patterned form. Advantageously, pattering is implemented by masking the part of the underlying surface of the liquid-soluble polymer that is not intended to be covered with polysiloxane with a protective mask film, like a PET or polyethylene (PE). The polysiloxane typically needs to be cured and the protective mask film is preferably removed before the polysiloxane is cured for the next deposition stage. As mentioned above, other methods for patterning, like slot die coating, may be applied without deviating from the scope of protection.

Finally, a layer of skin-adhesive is deposited (stage 308) on the second layer of polysiloxane. Also the layer of skin-adhesive patterned to include an opening that coincides with the sensing region. The layer of skin-adhesive can be deposited on the second layer of polysiloxane before the second layer of polysiloxane is deposited on the conductive layer, or the deposition can be implemented as a separate stage.

Roll-to-roll processing is a fabrication method used for manufacturing products. In a roll-to-toll process a web of materials can be continuously fed from one reel onto another and at the same time materials can added to the progressing web or removed from it to produce a desired product. As described with FIG. 3, by means of the defined layer structure a continuous stream of adhesive skin patches can be formed as an accumulating web in an industrial roll-to-roll process that progresses sequentially through the layer deposition stages described herein. As the roll-to-roll processing requires a certain robustness from the web that is reeled, the dual layer structure presented in FIG. 1C may be advantageous for such applications. By adjusting process parameters (like speed of the web) and material characteristics of the liquid soluble polymer 106, the processing method can be implemented without stage 301, i.e. start by depositing a first layer of polysiloxane on a film of liquid-soluble polymer.

As an alternative, the layered structure can be industrially fabricated in a sequentially progressing sheet-to-sheet printing process wherein the described deposition stages are performed progressively on a substrate sheet. As a more detailed implementation example, in a sheet-to-sheet process, a water-soluble polyvinyl alcohol (PVA) film can be formed (stage 300) on a PET carrier film by wire bar coating. Premixed two-component PDMS-based silicone elastomer is then deposited (stage 302) on a part of the PVA film by wire bar coating. Part of the PVA film is left exposed by masking the PVA film with a PET film and the masking PET film is removed before curing the silicone elastomer. In the next stage, a silver interconnect pattern is fabricated by screen printing (stage 304) partly on the silicon elastomer layer and partly on the exposed PVA layer. The part of the silver interconnect pattern on the exposed PVA then forms (when mounted on to the skin) an exposed pad for an external electrical connection. The silver interconnect pattern is then encapsulated (stage 306) by wire bar coating with another layer of the silicone elastomer. The area of skin electrode is left exposed by masking the silicone elastomer layer with a sheet of protective polyethylene (PE) liner (with low tack adhesive) film before the wire bar coating and removing the mask film before curing. A silver/silver chloride (Ag/AgCl) skin electrode is then fabricated (stage 304) on the exposed interconnect. Then a silicone based 2-component skin adhesive is deposited (stage 308) by wire bar coating. The silver/silver chloride skin electrode is masked with a protective polyethylene (PE) liner film that is removed after coating. Optionally, especially in commercial production, a protective film can be laminated on the skin adhesive layer in the same process.

FIG. 4 includes three images that show an exemplary skin patch formed with the layered structure described with FIGS. 1 and 2. The sample shows how well the ultra-thin skin patch conforms with the skin, even under stress ((i) compression, (ii) twist, (iii) stretch).

FIG. 5 illustrates an advantageous use case for an adhesive skin patch, formed with the layer structure described herein. FIG. 5 shows two items 500, 502 of the exemplary skin patch, explained in more detail with FIG. 2. Each of these skin patches 500, 502 include a skin electrode 504, 506 that is formed of the sensing region of the conductive layer in the layer stack. Each of these skin patches 500, 502 include also an external contact 508, 510 and a conductive path 512, 514 that electrically connects the external contact to the skin electrode. The external contacts and the conductive paths are also formed of the conductor region of the conductive layer in the layer stack. FIG. 5 shows also an interposing unit 504, a separate electrical device that can be connected to the external connects of the skin patches 500, 502. The interposing element 504 can be a more complex device that includes electrical components configured to process, store and/or output information based on the signals derived from the skin electrodes. The arrangement provides a very compact setup for, for example, electrocardiogram (ECG) monitoring.

In the earlier described examples, the conductor includes the conductive layer 110 that is between the first layer of polysiloxane 108 and the adhesive layer 114 or between the first layer of polysiloxane 108 and the second layer of polysiloxane 112. The sensing region 118 of the conductive layer 110 thus opens for skin contact, and the conductor region 116 opens directly for external contact through the recess 120 in the first layer of polysiloxane 108. However, the skin patch may also be a multi-layer structure that includes one or more conductors, wherein each conductor may be separated and thus electrically isolated from other conductors by at least one layer of polysiloxane. The conductors can be electrically connected by vias that extend through the one or more intermediate layers of polysiloxane. A via may extend through one separating layer of polysiloxane, or it may interconnect two conductive layers through two or more intermediate layers of polysiloxane. A via may in some configurations extend through one or more conductive layer patterns and thus interconnect more than two conductive layers.

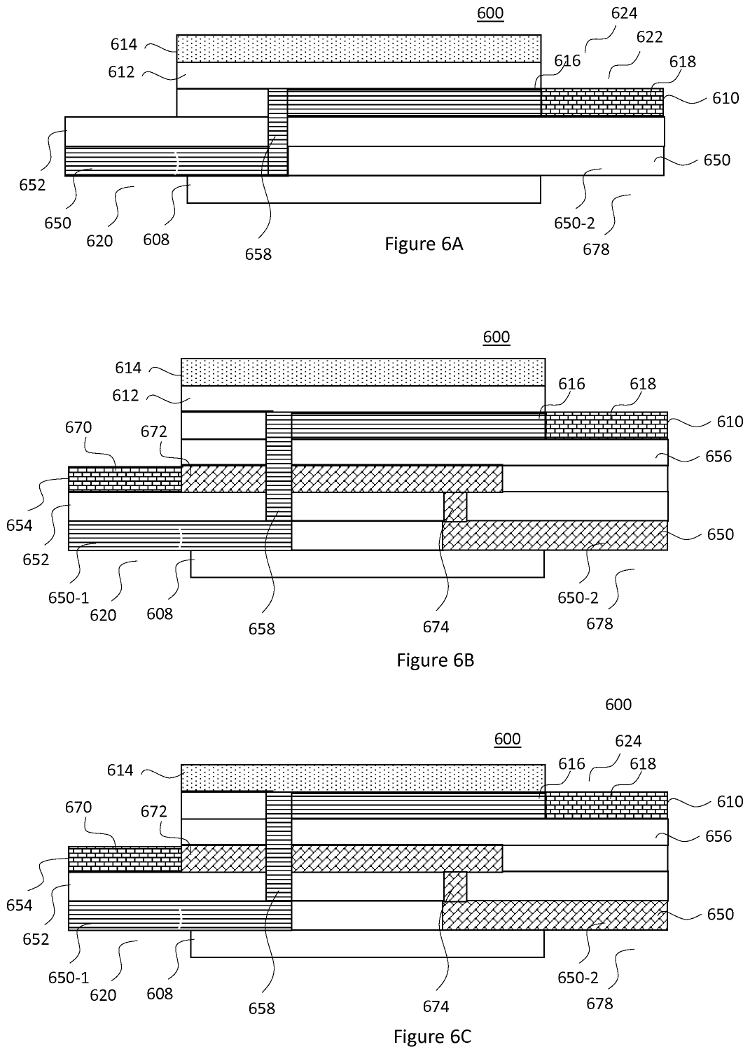

FIGS. 6A to 6C illustrate layers of an on-skin part 600 in exemplary multi-layer stacks for a skin patch. Elements that correspond with each other in FIGS. 1A to 1C and FIGS. 6A to 6C are denoted with similar reference numbers and additional description for those elements may be referred from description of FIGS. 1A to 1C. The on-skin part 600 of FIG. 6A corresponds to the embodiment of FIG. 1C and includes a first layer of polysiloxane 608, a first conductor in form of a first conductive layer 610, a second layer of polysiloxane 612, and an adhesive layer 614. The first conductive layer 610 is formed into a pattern that comprises a conductor region 616 and a sensing region 618 that is in electrical connection with the conductor region 616. In the following examples, however, the on-skin part of the multi-layer structure includes further layers between the first layer of polysiloxane 608 and the first conductive layer 610 of the first connector. The further layers in the example of FIG. 6A include a third layer of polysiloxane 652 and a second conductive layer 650, deposited on the first layer of polysiloxane 608 before deposition of the first conductive layer 610, in the order shown in FIG. 6A. The second conductive layer 650 is patterned into a form of at least one conductor. The first conductor is provided in the form of the first conductive layer 610 and includes the sensing region 618 and the conductor region 616. In use, the sensing region 618 comes in contact with the skin through the opening 622 in the second layer of polysiloxane 612 and opening 624 in the adhesive layer 614. However, here the opening 620 in the first layer of polysiloxane 608 does not open directly to the conductor region 616 of the first conductive layer 610, but to a second conductor provided by the second conductive layer 650. This second conductor is electrically connected to the conductor region 616 in the first conductive layer 610 by means of a via 658. Signals generated on the skin become thus accessible through the second conductive layer 650.

In the manufacturing process, a via between two conductive layers is formed by first patterning a recess to an intermediate layer or intermediate layers of polysiloxane between the conductive layers. When a conductive layer is deposited over a recess, it becomes filled with fluidic conductive material of the conductive layer and after curing forms the via. In the example of FIG. 6A, the via 658 is formed by patterning a recess that extends through the third layer of polysiloxane 652. The third layer of polysiloxane 652 is in this example patterned so that the recess for the via 658 extends through the third layer of polysiloxane 652, and when the conductor region 616 of the first conductive layer 610 is deposited, the fluidic conductive material fills the recess and the via 658 is formed.

Accordingly, in the example of FIG. 6A, a first conductor is provided by the first conductive layer 610 and a second conductor is provided by the second conductive layer 650. The via 658 connects electrically the first conductor and the second conductor. Accordingly, signals generated through skin contact of the first conductor (through the sensing region 618 of the first conductive layer 610) can be accessed through the second conductor (access to the conductive layer 650 through opening 620 in the first layer of polysiloxane 608). Alternatively, electrical signals may be fed on the skin through the second conductor.

The example of FIG. 6B illustrates a further option where said further layers between the first layer of polysiloxane 608 and the first conductive layer 610 include the second conductive layer 650, the third layer of polysiloxane 652, described with FIG. 6A, and also a third conductive layer 654 and a fourth layer of polysiloxane 656. These layers are deposited on the first layer of polysiloxane before the first conductive layer 610 and in the order shown in FIG. 6B.

In the example of FIG. 6B, the second conductive layer 650 is formed into a pattern that includes two separate layer parts 650-1 and 650-2. These parts 650-1 and 650-2 become electrically isolated by deposition of the third layer of polysiloxane 652 and thus form two separate conductors. A third conductor can thus be provided by the second part 650-2 of the second conductive layer 650. A fourth conductor can be provided by the third conductive layer 654, which includes a sensing region 670 and a conductor region 672. The sensing region 670 of the third conductive layer 654 becomes exposed to the skin through openings in the adhesive layer 614, the second layer of polysiloxane 612 and the fourth layer of polysiloxane 656. Due to the multi-layer structure, the adhesive patch can thus include skin electrodes in various locations in the overall surface area of the patch. As shown in FIG. 6B, the conductor region 672 of the third conductive layer 654 may be separately connected to the part 650-2 of the second conductive layer 650 by means of the via 658. Accordingly, signals generated through skin contact of the fourth conductor (through sensing region 670 of the third conductive layer 654) can be accessed externally through the third conductor (access to the part 650-2 through opening 678 in the first layer of polysiloxane 608). Alternatively, electrical signals may be input through the third conductor and become fed on the skin through the fourth conductor.

FIG. 6C illustrates an example that corresponds with the example of FIG. 1A in that the adhesive layer 614 is patterned to include the opening 624 to the sensing region 618 of the first conducting layer 610, and the structure does not include the second layer of polysiloxane 612 shown in FIG. 1C or 6A and 6B. In layers below the first conductive layer 610, the stack incudes layers described with the example of FIG. 6B.

As discussed, the vias can extend through layers of polysiloxane without crossing. This means that the described stack can provide various combinations of input and output interfaces in freely selected locations in the skin patch. The multi-layer form enables dense packaging of electronics and thereby more complex circuits and operational functions. Due to the inventive use of fluids that can fill recesses is earlier cured forms and can also be patterned and cured to form recesses, the overall structure still remains extremely thin, even when stacked into multi-layer form. Accordingly, when the patch is adhesively attached on skin, it conforms with wrinkles, contours and elastic motions of the skin so that disturbance caused by measurements implemented with the skin patch are minimised.

As can be seen in FIGS. 6A-6C, the conductors provided by the conductive layers are patterns that do not necessarily cover the whole underlying surface area. In Figures, boxes without reference numbers in the layer stacks illustrate the amounts of polysiloxane or adhesive material that due to the wet stage of the deposition process fills at least part of the regions not covered by an underlying pattern of conductive layer.

FIGS. 7A and 7B illustrate further examples for an on-skin part 700 in a multi-layer stack for a skin patch. Elements that correspond with each other in FIGS. 1A to 1C, FIGS. 6A to 6C and FIGS. 7A to 7B are denoted with similar reference numbers and additional description for those elements may be referred from description of FIGS. 1A to 1C and from FIGS. 6A to 6C.

The example of FIG. 7A corresponds with the example described with FIG. 6B. As in FIG. 6B, the on-skin part 700 includes a first layer of polysiloxane 708, a first conductor provided by a first conductive layer 710, a second layer of polysiloxane 712 and an adhesive layer 714. The first conductive layer 710 is formed into a pattern that comprises a conductor region 716 and a sensing region 718 that is in electrical connection with the conductor region 716. Also in this example, the further layers of the multi-layer structure include a second conductive layer 750, a third layer of polysiloxane 752, and a fourth layer of polysiloxane 756, in the order shown in FIG. 7A. The second conductive layer 750 is again patterned into a form of two separate layer parts 750-1 and 750-2 of conductive material. These parts 750-1 and 750-2 become electrically isolated by deposition of the third layer of polysiloxane 752 and thus form two separate conductors, a second conductor and a third conductor.

However, this time the fourth conductor is provided by two overlapping conducting layers, a third conductive layer 780 and a fourth conductive layer 782. The third conductive layer 780 provides the conductor region of the fourth conductor and the fourth conductive layer 782 provides the sensing region of the fourth conductor. The third conductive layer 780 is deposited as a pattern on the third layer of polysiloxane 752 and the fourth conductive layer 782 is deposited as a pattern on the third conductive layer 780. The fourth layer of polysiloxane 756 is deposited on top of the third conductive layer 780 and the fourth conductive layer 782 during its wet deposition stage fills void spaces in the underlying third and fourth conductive layers 780, 782. The third layer of polysiloxane 754 and the fourth layer of polysiloxane 756 are patterned to include a recess for the via 758 that becomes filled with conductive material at deposition of the conductor region 716 of the first conducting layer 710. The third layer of polysiloxane 754 is also patterned to include a recess for the via 774 that becomes filled with conductive material at deposition of the the third conducting layer 780. The second and fourth layer of polysiloxane 712, 756 and the adhesive layer 714 that are deposited after the fourth conductive layer 782 are patterned to include a recess that provides an opening to at least part of the conductive layer 782 of the fourth conductor.

Accordingly, in the example of FIG. 7A, signals generated through skin contact of the first conductor (through the sensing region 718 of the first conductive layer 710) can be accessed externally through the second conductor (access to the part 750-1 through opening 720 in the first layer of polysiloxane 708). Alternatively, electrical signals may be input through the second conductor and fed on the skin through the first conductor. The third conductor is provided by the second part 750-2 of the second conductive layer 750 and the fourth conductor is provided by the combination of the third conductive layer 780 and the fourth conductive layer 782. Signals generated through skin contact of the fourth conductor (through the sensing region provided by the third conductive layer 782) can be accessed externally through the third conductor (access to the part 750-2 through opening 778 in the first layer of polysiloxane 708). Alternatively, electrical signals may be input through the third conductor and fed on the skin through the fourth conductor.

In the example of FIG. 7A, the fourth layer of polysiloxane 756, the second layer of polysiloxane 712 and the adhesive layer 714 are formed on top of the third layer of polysiloxane 752 and expose the sensing region 782 of the fourth conductor in a position where it is surrounded by the adhesive layer. This means that a firm connection to the skin around the third connector 780 is ensured.

FIG. 7B illustrates an example that corresponds with the example of FIG. 1A or FIG. 6C in that the structure does not include the second layer of polysiloxane 712 shown in FIG. 7A. In layers below the first conductive layer 710, the stack incudes layers described with the example of FIG. 7A.

The examples described herein are schematic and non-restrictive illustrations of elements and terms necessary to disclose the invention. For example, conductive layers that in some examples provide both the sensing region and the conductor region may be replaced by two conductive layers wherein one conductive layer provides the sensing region and the other one provides the conductor region, and vice versa. The example stacks of FIGS. 6A to 6C and 7A to 7C show stacks with and without the second layer of polysiloxane, but one or more additional layers of polysiloxane or other suitable materials can be included in the stack as well. For a person skilled in the art, it is clear that the described structures and deposition stages have various possible implementation options that are not explicitly shown here but are covered by the scope of the appended claims.

1. An adhesive skin patch including a first layer of polysiloxane, a first conductor and an adhesive layer; wherein
the first conductor comprises a conductor region and a sensing region that is in electrical connection with the conductor region;
the first layer of polysiloxane includes an opening to at least part of the conductor region of the first conductor or to a further conductive layer electrically connected to the conductor region of the first conductor by one or more vias;
the adhesive layer includes an opening to at least part of the sensing region of the first conductor.

2. An adhesive skin patch according to claim 1, wherein the first conductor is a conductor layer formed into a pattern that comprises the conductor region and the sensing region.

3. An adhesive skin patch according to claim 1, wherein the polysiloxane includes polydimethylsiloxane.

4. An adhesive skin patch according to claim 1, wherein the part of the conductor region of the first conductor is formed to provide a contact for electrical connection with the sensing region.

5. An adhesive skin patch according to claim 1, wherein
the adhesive skin patch includes a second layer of polysiloxane between the first conductor and the adhesive layer; and
the adhesive layer and the second layer of polysiloxane include a common opening to at least part of the sensing region.

6. An adhesive skin patch according to claim 5, wherein in regions beyond the openings, the first layer of polysiloxane and the second layer of polysiloxane enclose the first conductor in water-tight manner.

7. An adhesive skin patch according to claim 1, wherein
the adhesive skin patch includes one or more further conductors, wherein each conductor is separated from another conductor by one or more intermediate layers of polysiloxane;
the conductors of the adhesive skin patch are electrically connected by vias that extend through one or more intermediate layers of polysiloxane.

8. An adhesive skin patch according to claim 7, wherein at least one of the vias interconnects two conductors through two or more intermediate layers of polysiloxane.

9. An adhesive skin patch according to claim 1, wherein the skin patch includes a releasable part.

10. An adhesive skin patch according to claim 9, wherein the releasable part includes a layer of liquid-soluble polymer.

11. An adhesive skin patch according to claim 10, wherein the liquid-soluble polymer is soluble with water.

12. A method for creating a layer structure for adhesive skin patches, the method including:
depositing on a layer of liquid-soluble polymer a first layer of polysiloxane that is patterned to include an opening and cured to function as an elastic solid;
depositing on the first layer of polysiloxane a first conductor that includes a conductor region and a sensing region that is in electrical connection with the conductor region or depositing on the first layer of polysiloxane a second conductor, at least one intermediate layer of polysiloxane and a first conductor that includes a conductor region and a sensing region, wherein the conductor region of the first conductor or the second conductor is aligned to at least partly coincide with the opening in the first layer of polysiloxane;
depositing on the first conductor a layer of skin-adhesive patterned to include an opening that coincides with the sensing region of the first conductor.

13. A method according to claim 12, wherein the method includes providing the first conductor as a conducting layer formed into a pattern that comprises the conductor region and the sensing region.

14. A method according to claim 12, wherein the method includes depositing on the first conductor a second layer of polysiloxane that is patterned to include an opening that coincides with the opening that is patterned to the layer of skin-adhesive and coincides with the sensing region.

15. A method according to claim 12, wherein the method includes depositing the layer of liquid-soluble polymer first on a surface of a sheet of support substrate.

16. A method according to claim 12, wherein the layer structure is created in a roll-to-roll process wherein the layer structure is formed of a web of the roll-to-roll process that progresses sequentially through the deposition stages.

17. A method according to claim 12, wherein the layer structure is created in a sheet-to-sheet printing process wherein the layer structure is formed on a support substrate sheet that is processed in a progressing sequence through the deposition stages.

18. A method according to claim 12, wherein the deposition of the first conductor is performed in two parts wherein the conductor region is deposited before the deposition of the second layer of polysiloxane, and the sensing region is deposited before or after the deposition of the second layer of polysiloxane, into the opening in the second layer of polysiloxane.

19. A method according to claim 12, wherein the method includes
depositing the second conductor in form of a second conductive layer;
depositing on the second conductive layer one or more intermediate layers of polysiloxane;
patterning the one or more intermediate layers of polysiloxane with a recess;
depositing the first conductor so that the recess becomes filled with conductive material thereby forming a via that electrically connects the first conductor to the second conductor.

20. A method according to claim 19, wherein the method includes
depositing the second conductor as part of a second conductive layer that includes the second conductor and a third conductor;
depositing on at least one of the intermediate layers of polysiloxane a fourth conductor that includes a conductor region and a sensing region that is in electrical connection with the conductor region
patterning intermediate layers of polysiloxane between the third conductor and the fourth conductor with a recess;
depositing the fourth conductor so that the recess becomes filled with conductive material thereby forming a via that electrically connects the third conductor to the fourth conductor.
